(19) United States
(12) Patent Application Publication
(10) Pub. No.: US 2026/0002947 A1
(43) Pub. Date: Jan. 1, 2026

(54) METHODS, SYSTEMS AND COMPOSITIONS FOR TRAUMATIC BRAIN INJURY AND ASSOCIATED NEURODEGENERATIVE DISEASE IMMUNE RESPONSE DIAGNOSTICS

(71) Applicant: NeuroTheranostics, Inc., Detroit, MI (US)

(72) Inventors: John Anagli, Detroit, MI (US); Tracy Anagli, Detroit, MI (US)

(73) Assignee: NeuroTheranostics, Inc., Detroit, MI (US)

(21) Appl. No.: 19/322,337

(22) Filed: Sep. 8, 2025

Related U.S. Application Data
(63) Continuation-in-part of application No. 17/718,713, filed on Apr. 12, 2022.
Application No. 17/718,713 is a continuation-in-part of application No. 16/534,098, filed on Aug. 7, 2019, now abandoned.
(60) Provisional application No. 62/715,353, filed on Aug. 7, 2018.

(51) Int. Cl. G01N 33/68; G01N 33/543

(52) U.S. Cl. CPC G01N 33/6896; G01N 33/54386; G01N 2800/28

(57) ABSTRACT

A system and method for diagnosing traumatic brain injury (TBI) includes a proteome biochip including a set of brain protein fractions including primary serum autoantibodies reactive with brain protein autoantigens released by the TBI printed into micro-wells of a glass slide. The biochip hybridized with non-TBI and TBI-injured serum samples from which an autoantibody response profile is generated. The system additionally including labeled IgG or IgM secondary antibodies for addition to the micro-wells for binding with one of the primary serum autoantibodies, a side illumination laser to read the micro-wells in which the labeled IgG or IgM secondary antibodies are bound with one of the primary serum autoantibodies, and a readout detection system for the set of brain protein fractions to screen for autoantigens present in the micro-wells that contain the labeled IgG or IgM secondary antibodies bound with one of the primary serum autoantibodies to generate a heat map.

### RELATED APPLICATIONS

This application claims is a continuation-in part of U.S. Utility application Ser. No. 17/718,713, filed 12 Apr. 2022; that in turn is a continuation-in part of U.S. Utility application Ser. No. 16/534,098, filed 7 Aug. 2019; which claims priority benefit of U.S. Provisional Application Ser. No. 62/715,353, filed 7 Aug. 2018; the contents of which are hereby incorporated by reference.

### FIELD OF THE INVENTION

The present invention generally relates to compositions, systems, and methods for diagnosing traumatic brain injury and associated neurodegenerative disease immune response, and more specifically to systems biology-based whole brain traumatic brain injury proteome biochip and autoimmune response biomarker panels to detect, measure and track the onset and progression of traumatic brain injury and associated neurodegenerative diseases.

### BACKGROUND

Traumatic brain injury (TBI) occurs in mammals, including in humans, when a sudden trauma causes damage to the brain. TBI can result when the head suddenly and violently hits an object, blast-induced neurotrauma can result from the blast waves without impact with a physical object or when an object pierces the skull and enters brain tissue. Symptoms of TBI include a loss of consciousness, headache, confusion, lightheadedness, dizziness, blurred vision or tired eyes, ringing in the ears, bad taste in the mouth, fatigue or lethargy, a change in sleep patterns, behavioral or mood changes, trouble with memory, concentration, attention, or thinking, vomiting or nausea, convulsions or seizures, an inability to awaken from sleep, dilation of one or both pupils of the eyes, slurred speech, weakness or numbness in the extremities, loss of coordination, and increased confusion, restlessness, and agitation. Symptoms of a TBI can be mild, moderate, or severe, depending on the extent of the damage to the brain.

TBI is a leading cause of death and disability in the United States. TBI is often referred to as the silent epidemic because the problems that result tend to be emotional and cognitive in nature (e.g., impaired memory, change in character traits, difficulty concentrating), not just physical¹⁻³. While TBI can occur as a result of auto accidents, falls, violence, or sports injuries it has come out of the shadows with the war in Iraq and Afghanistan where 20% or more of the causalities are due to head injuries. However, the civilian population has been suffering from this affliction in larger numbers for years. In the United States alone approximately 2.5 million people annually suffer from a head injury, from a mild bump that will heal over time to ones so severe the patient will die^{2,3}. About 52,000 patients each year die from a TBI. Between 75 and 90% of TBIs are mild or moderate (MTBI) and are challenging to diagnose^{1, 3-5}. Furthermore, many sufferers fail to recognize the potential severity and seriousness of their injury and do not seek medical attention². Thus, TBI is under-diagnosed and under-represented in medical statistics. However, even brief alterations in mental status can inflict profound and persistent impairment of physical, cognitive, and psychosocial functioning^{6,7.}

Persons experiencing a single or repetitive TBI are at increased risk for neurodegenerative disorders such as chronic traumatic encephalopathy (CTE)⁸⁻¹¹, Alzheimer's disease (AD)¹²⁻¹⁴ and Parkinson's disease (PD)¹⁴⁻¹⁵. Some patients show an early and dramatic decline in function (CTE). However, CTE is a neuropathological disorder for which clinical diagnostic criteria are lacking, that is largely confined to professional athletes or military personnel, and for which the incidence rates and societal costs are unknown.

By contrast the greater health risk is among the larger number of patients with mild TBI (MTBI) who appear to recover from their injury within a few months, remain stable for decades, only to show a late decline (e.g., PD; AD). It is estimated that 20% of persons will develop PD or AD during their lifetime; the annual costs of these disorders in the US is presently ˜$250 billion, and is expected to exceed $1 trillion by 2050. The risk for PD or AD following MTBI is doubled, and possibly greater with repeated or more severe head injuries. Yet there is no reliable way to determine which TBI cases will progress to develop these common neurodegenerative disorders, and the mechanisms by which TBI leads to neurodegeneration are poorly understood. Furthermore, there is no reliable way to determine if a patient had a “prior” TBI (head trauma one or more years ago), apart from recall (which may measure exposures or their severity inaccurately). Indeed, the annual incidence of TBI far outnumbers that of stroke (795,000 per year)¹⁶, heart attack (735,000)¹⁶, breast cancer (231,840 per year)¹⁷, HIV/AIDS (47,500 per year)¹⁸ or spinal cord injury (12,000) (FIG. 1). However, unlike stroke where victims are predominantly over the age of 65 years, TBI inflicts all age groups with a significant peak during adolescence and early childhood¹⁹, the age group which has the greatest economic potential. The physical, social, and economic sequelae of brain injury can persist for the victim's entire life, a burden that is borne by the individual and by society. According to the Center for Disease Control and Prevention (CDC), the annual estimated direct and indirect cost of TBI is close to $76.5 billion in the United States^{3,20}. Importantly, TBI is being recognized more as a disease process, rather than a discrete event, because of the potential it presents for non-reversible and chronic health effects and as a model of neurodegenerative diseases generally^{21,22.}

Brain injury activates the immune system to produce autoantibodies against specific brain proteins [23, 24]. The autoimmune response to TBI is triggered by immune surveillance of brain proteins previously sequestered by the blood-brain barrier and also by the generation of injury-induced chemical modifications that increase the antigenicity of brain proteins. While the mechanisms involved are not fully understood, critical features underlying the immune response to brain proteins include: a weakening of the blood brain barrier, invasion of immune cells into the injured tissue; and the subsequent reaction of these cells with specific proteins and posttranslational modifications, resulting in the expression of autoantibodies. The appearance of circulating autoantibodies has been observed following TBI. For example, glial fibrillary acidic protein (GFAP). Zhang et al Human traumatic Brain Injury induces autoantibody response against glial fibrillary acidic protein and its breakdown products. is proteolyzed and endothelial nitric oxide synthase (eNOS) is phosphorylated following a TBI, rendering these post-translationally modified proteins more autoantigenic than their intact forms.

A growing body of experimental evidence is now confirming that some of the specific proteins targeted by autoantibodies are shed into the general circulation, where they may be evaluated as biomarkers. It is likely, therefore, that the presence of these autoantigens in blood precedes the more delayed appearance of their respective autoantibodies, due to the time required to marshal a humoral immune response. Autoantigen microarrays have previously been used for multiplex characterization of autoantibody responses²⁶. For example, autoantigen microarrays have been used to profile autoimmune responses to multiple sclerosis²⁷⁻²⁸, lupus erythematosis²⁹⁻³², primary graft dysfunction after lung transplantation³³ and cancer³⁴. Biochip technology is being developed by a number of researchers and companies to provide a standard near point-of-care diagnostic system that can be used in hospitals and other laboratories as well as in the field and would save time and money compared to current systems, which require samples to be sent to a centralized lab for confirmatory diagnosis. Cancer, infectious disease and respiratory syndrome chips are under current development. However, a systems biology-based whole brain traumatic brain injury proteome microarray biochip platform designed to detect and measure autoimmune response biomarker signature panels that track the onset and progression of TBI and other neurological diseases has not been explored.

Accordingly, there exists a need in the art for a standard near point-of-care diagnostic system that can be used in hospitals and other laboratories as well as in the field to diagnose and track the onset and progression of TBI and associated neurodegenerative disease immune response and other neurological diseases.

### SUMMARY OF THE INVENTION

A proteome biochip for diagnosing traumatic brain injury (TBI) includes brain protein fractions printed into a plurality of micro-wells of a glass slide. The proteome biochip is helpful in determining the details of a traumatic brain injury.

A method to diagnosing traumatic brain injury is also provided that includes a proteome biochip being provided. A biological sample from a patient is then applied to the biochip. By determining binding of sample proteins from the patient, the nature of the traumatic brain injury is diagnosed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the present invention will now be described, by way of example only and without disclaimer of other embodiments, with reference to the accompanying drawings:

FIG. 1 is a prior art plot that shows the annual incidence of traumatic brain injury in the United States, compared to other serious, debilitating diseases;

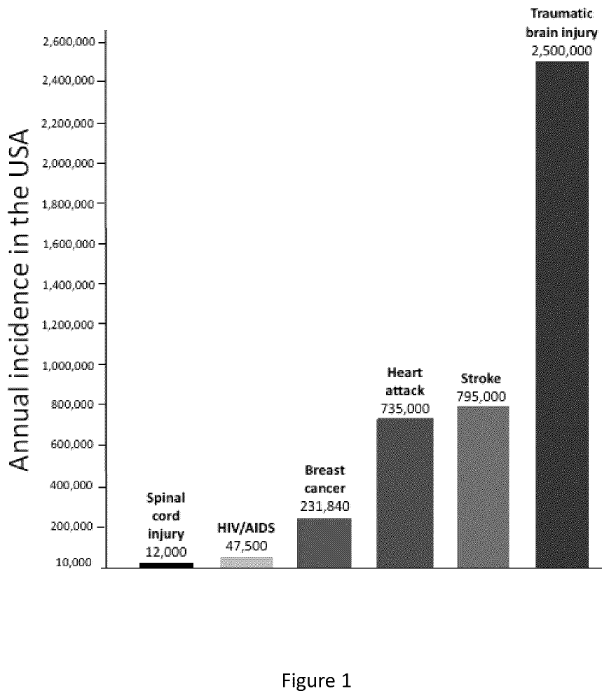

FIG. 2 illustrates the inventive whole brain TBI proteome microarray biochip platform; and

FIG. 3 is a heat map comparing the proteomic profiles of subcellular protein fractions derived from a whole brain TBI proteome model.

### DETAILED DESCRIPTION

The present invention has utility as methods, systems, and compositions for diagnosing and tracking the onset and progression of traumatic brain injury and associated neurodegenerative disease immune response. The present invention further has utility as a fast and inexpensive standard near point-of-care diagnostic system that can be used in hospitals and other laboratories as well as in the field.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

As used herein the term “diagnosing” means recognizing the presence or absence of a neurological or other condition, such as an injury or disease. Diagnosing is in a subset of circumstances referred to as the result of an assay wherein a particular ratio or subset of proteins detected or is absent.

As used herein an injury is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An event is illustratively a physical trauma, such as an impact (percussive), or a biological abnormality, such as a stroke resulting from either blockade or leakage of a blood vessel. An event is optionally an infection by an infectious agent. A person of skill in the art recognizes numerous equivalent events that are encompassed by the terms injury or event.

An injury is in some instances a physical event, such as a percussive impact. An impact is the like of a percussive injury, such as resulting from a blow to the head that either leaves the cranial structure intact or results in breach thereof. Experimentally, several impact methods are used illustratively including controlled cortical impact (CCI) at a 1.6 mm depression depth, equivalent to severe TBI in humans. This method is described in detail by Cox, C D, et al., J Neurotrauma, 2008; 25(11):1355-65. It is appreciated that other experimental methods producing impact trauma are similarly operable.

The present disclosure provides a whole brain proteome microarray biochip platform that displays brain proteomes (i.e. all the proteins expressed in the brain at a specific time, that is a particular point in time) and provides the ability to screen for patient autoantibody biomarkers and therapeutic targets, as well as provide the ability to characterize TBI-associated neurological disease states.

Biochips, also referred to as microarrays or biologic microchips, are used to analyze many parameters of a sample in parallel. Depending on the nature of the probes that are immobilized in the microarray elements, various tasks can be implemented, including the detection of specific nucleic acid sequences and their mutations, the study of certain gene expressions, immunologic analyses, infectious agent detection, etc. Frequently, an analyzed sample is labeled with a fluorescent dye, and the fluorescence intensity of the various microarray elements after their incubation with the sample indicates the affinity of the sample to the molecular probes immobilized in the microarray elements.

The system and method of the present disclosure are capable of surveying potential autoimmune responses to all brain proteins. The present disclosure utilizes over two dozen identified TBI-induced brain proteome-specific autoimmune response chronic biomarker hits that are validated for use in biomarker quantitative assessments on a diagnostic biochip platform to track the progression of neurodegeneration following TBI. The inventive brain proteome chip has also confirmed that a number of brain-specific proteins as well as their post-translationally modified forms are released into the general circulation following TBI and become targets of the autoimmune response. Further, a subset of the TBI autoantigens revealed by the biochip are targets of TBI drug candidates being developed at NeuroTheranostics. These TBI drug target autoantigens and their corresponding autoantibodies serve as pharmacodynamics biomarkers for theranostic TBI drug development. The compositions, systems, and methods of the present disclosure provide knowledge of the autoimmune mechanisms involved in TBI helpful in identifying pathways of injury and biomarkers for the surveillance and diagnosis of TBI-associated neurodegenerative diseases such as CTE, AD and PD. This information provides a framework for addressing chronic neuro-immune mechanisms that may impair neural regeneration and restoration of normal neuronal functions. Moreover, this comprehensive study of brain proteins potentially generating chronic biomarkers of autoimmune responses optimizes chances of detecting relevant biomarkers that can be used to guide therapy development.

Without limitation to only those embodiments expressly disclosed herein and without disclaiming any embodiments, some embodiments of the invention comprise methods, systems, and compositions to selectively detect and measure TBI proteome-specific autoimmune response biomarker signature panels, that is, panels of autoantigens released into the general circulation of a patient following TBI and that become targets of the autoimmune response and/or that are specific for TBI-induced neurodegenerative disease. In some embodiments, such trauma injured brain proteome-specific autoimmune response biomarker signature panels may be used to track the onset and progression of TBI and other neurological diseases in a patient by measuring in the blood, plasma, serum, saliva, or urine of the patient with a chronic TBI biomarker profile or signature. A biological sample in some embodiments is a fluid in communication with the nervous system of the subject prior to being isolated from the subject (e.g., CSF or blood), or is brain tissue.

According to embodiments of the present disclosure, systems biology-based, trauma injured whole brain proteome models are provided. Additionally, embodiments of the present disclosure provide isolated and/or made intact, post-translationally modified and fragmented versions of brain proteome-specific autoantigens printed onto a biochip to detect, measure and track the onset and progression of TBI and associated neurodegenerative diseases.

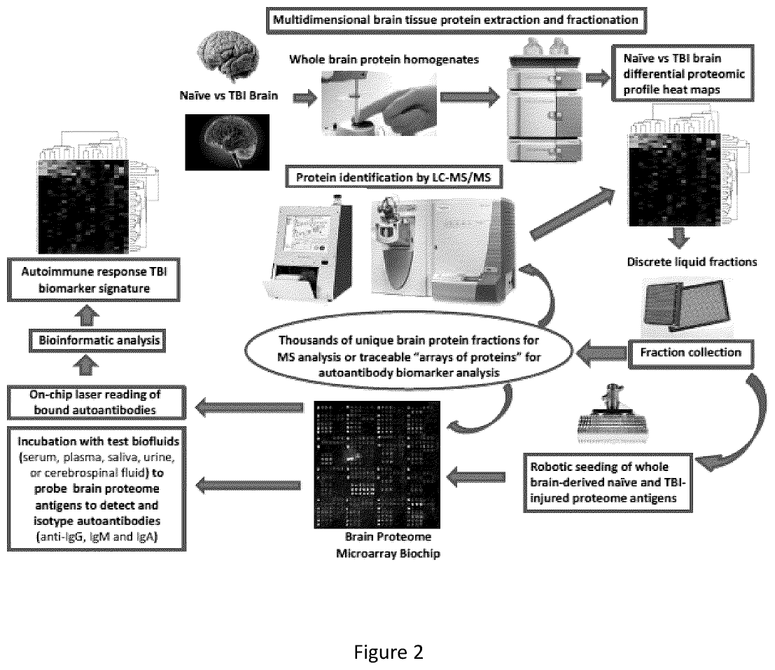

Central to the overall strategy is the use of a rat controlled cortical impact (CCI)-or blast-induced traumatic brain injury model to create naïve, trauma injured non-treated and trauma injured TBI drug-treated brain proteomes that are subsequently subjected to a multidimensional separation process to fractionate the thousands of proteins that potentially may be present in the brain at any given time (ex. control non-TBI and TBI brain proteomes) (FIG. 2). Using buffers and subcellular fractionation techniques, whole brain proteins are first extracted as “soluble” and “particulate” proteins. Then the soluble and particulate brain proteins are fractionated and enriched by multi-dimensional protein separation methods such as high performance liquid chromatography, consisting of a combination of chromatofocusing, hydrophobic interaction, ion exchange, size exclusion, affinity (antibody-based) or reversed-phase chromatography. This all-liquid phase protein fractionation typically generates thousands of unique protein fractions per sample of soluble and particulate proteins which are isolated into multi-titer well plates.

The fractionated TBI-injured and non-TBI rat brain proteins are identified by mass spectrometry and database search, and subsequently “spotted” onto a high-density whole brain proteome biochip for on-chip serological analyses of patient autoantibody biomarkers that can be used to diagnose and track the progression of TBI and TBI-related long-term neurological problems. To create the whole brain proteome chips, the brain protein fractions collected from primary serum samples having autoantibodies reactive with brain protein autoantigens released into the general circulation of a patient following TBI and that become targets of the autoimmune response are “printed” into the micro-wells of a glass slide or nitrocellulose-coated glass slide (biochip) by employing a Microarray Printer. Serum autoantibody responses to brain-specific proteins after TBI in humans have been identified. TBI autoantibodies show predominant immunoreactivity against a cluster of bands from ₃₈₋₅₀ kDa on human brain immunoblots, which are identified as Glial Fibrillary Acidic Protein (GFAP) and GFAP breakdown products. It has been found that GFAP autoantibody levels increase by 7 days after injury, and were of the IgG subtype predominantly, as disclosed by Zhang et al.³⁹

According to inventive embodiments, brain protein fractions having autoantibodies reactive with brain protein autoantigens released into the general circulation of a patient following TBI and that become targets of the autoimmune response include serum autoantibody responses to brain-specific proteins after TBI in humans

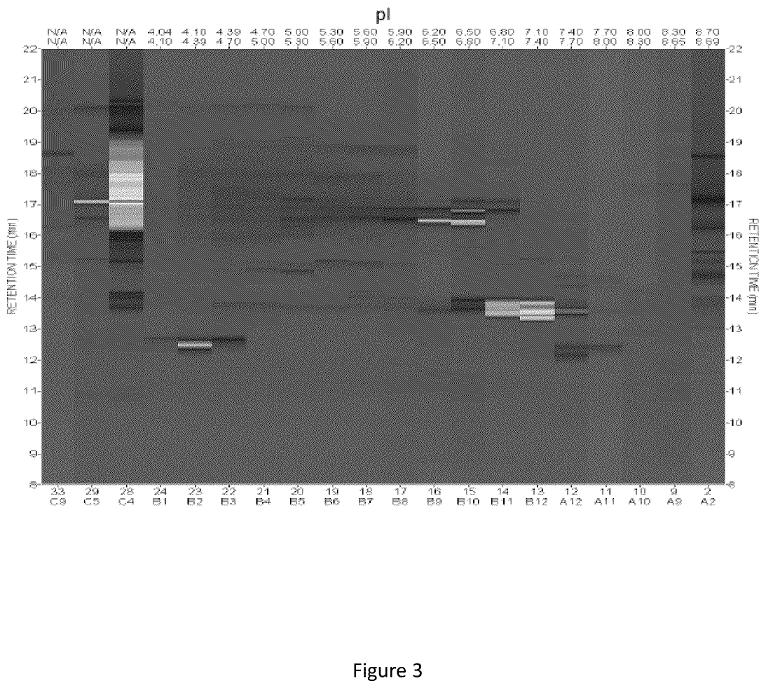

The brain proteome chips are then hybridized with control non-TBI (that is, serum samples collected from human subjects that have not experienced a TBI) and TBI-injured human serum samples (that is, serum samples collected from human subjects that have experienced TBI) from which a humoral (autoantibody) response profile is generated. After the serum sample containing the autoantibodies to be tested is applied, a labeled IgG or IgM secondary antibody is added to the biochip. These labeled IgG or IgM secondary antibodies are configured to and are able to bind with one of the primary serum antibodies. After the labeled IgG or IgM secondary antibodies are applied to the biochip and bind with one of the primary serum antibodies thereon, the biochip is then put in a reader and scanned using side illumination laser technology to detect reaction sites. A readout detection system can either be optical (fluorescence, chemiluminescence) or colorimetric. Such readout detection systems for use with biochips for diagnostic purposes are known in the art as evidenced by Lysov Y., Barsky V., Urasov D., Urasov R., Cherepanov A., Mamaev D., Yegorov Y., Chudinov A., Surzhikov S., Rubina A., et al. Microarray analyzer based on wide field fluorescent microscopy with laser illumination and a device for speckle suppression. Biomed. Opt. Express. 2017; 8:4798-4810. A readout detection system, also known as a microarray analyzer obtains images and measures the fluorescence intensity of microarrays at various wavelengths. Such analyzers contain lasers to excite fluorescence, barrier filters, optics to project images on an image detector, and a device for suppressing laser speckles on the microarray support. The speckle suppression device contains a fibre-optic bundle and a rotating mirror positioned in a way to change the distance between the bundle butt and mirror surface during each mirror revolution. Such analyzers provide for measurements with accuracy within ±5%. Obtaining images at several exposure times allows a significant expansion in the range of measured fluorescence intensities. Such analyzers are useful for high throughput analysis of the same type of microarrays. Such analysis results in heat maps comparing the proteomic profiles of subcellular protein fractions derived from a whole brain TBI proteome model, such as that shown in FIG. 3. The heat map indicating identified TBI-induced brain proteome-specific autoimmune response chronic biomarker hits that are only present following TBI, and thus allowing for diagnosis and quantitative assessments of TBI. The inventive brain proteome chip has also confirmed that a number of brain-specific proteins as well as their post-translationally modified forms are released into the general circulation following TBI and become targets of the autoimmune response.

The gel drops are in known positions on the brain proteome chip so when a serum sample reacts with them, the reaction positions could be detected and traced back to the previously mass spectrometry characterized protein fractions that are used to print the microarray. The candidate brain protein autoantigen(s) present in each gel drop could then be identified. Further characterization of the brain proteome antigen-serum autoantibody interaction can be accomplished with on-chip matrix-assisted laser desorption/ionization-time of flight mass spectrometry (MALDI-TOF MS). Here, the biochip comprising the high-density array of brain protein fractions as capture molecules for the serum autoantibodies directly interfaces with the MALDI mass spectrometer. Alternatively, a random peptide library is spotted on a biochip to discover brain injury specific autoimmune peptide antigens.

The present disclosure provides two types of whole brain TBI proteome specific biochips (i.e. profiling and diagnostic chips). Profiling biochips are produced using post-mortem: 1) whole brain proteome; and 2) brain tissue samples taken from specific brain regions of control non-TBI and TBI subjects. Following the identification of panels of autoantigens specific for TBI-induced neurodegenerative disease, diagnostic brain protein biochips composed of condensed arrays of known TBI-induced autoantigens and internal calibration standards are created for the quantitative determination of the autoimmune response biomarker levels.

### Materials and Methods

### Brain Sample Prep:

Soluble and particulate whole brain tissue protein extracts are diluted to 2.5 cc with ProteoSep Start Buffer (SB) (Eprogen) and buffer exchanged with 3.5 cc of SB using a PD-10 column (GE Healthcare) according to manufacturer's instructions. The PD-10 eluent is diluted to 5.5 cc with SB for injection onto the ProteoSep Chromatofocussing (CF) column.

Two-dimensional liquid chromatography: Using a ProteomeLab PF2D HPLC instrument, a chromatofocussing (CF) column is equilibrated with SB at 0.3 mL/min until the pH of the eluent was 8.5±0.01. 5.0 mL of the prepped sample is injected onto the CF column at 0.2 mL/min and the CF column is flushed with SB for 35 minutes with fraction collection (to capture the very basic proteins) into a 2.0 mL deep well plate. ProteoSep Eluent Buffer (EB) having a pH 4.0±0.1 is then introduced and fractions collected every 0.3 pH units starting with pH 8.3 until the pH remains stable at the final EB pH. A 1M NaCl solution containing 0.2% n-ctyl glucoside is introduced and fractions are collected containing the high acidic proteins.

Using a ProteoSep High Performance Reverse Phase (HPRP) HPLC column the fractions collected from the CF separation (˜20) are fractionated further in a second dimension under the following conditions:
Mobile Phase A: H₂O/0.1% TFA; Mobile Phase B: Acetonitrile/0.08% TFA
Gradient: 0% B for 2 min, 0-100% B in 30 min, 100% B for 2 min, 100-0% B in 2 min, 0% B 8 min (reequilibration)
Temperature: 50° C.
Flow rate: 750 μL/min
Detection: UV 214 nm

Fractions from the HPRP analysis are collected into 0.65 mL/well 96 well plates (Orochem) using a Gilson FC 203 fraction collector from 8-22 minutes @ 0.29 min/well. Each well contains 218 μL/well (48 wells total are collected per fraction). The plates are covered with foil sealing tape (3M) stored at −80° C. until further use.

### Microarray Preparation:

50 μL of a 40% glycerol (Sigma Ultrapure) water solution is added to each well of 96-well plates and then evaporated down to a final volume of 20 μL using a SpeedVac (ThermoFisher). 30 μL of PBS is then added to each well to make a 40% glycerol/PBS print buffer and the contents of each well transferred into 2 sets of 384 well plates for microarray printing. A solid (150 μm) pin QArray2 (Genetix, Ltd.) is used to spot the arrays on glass PATH slides (Grace Biosciences, Inc.). Each well is double spotted and sets of arrays of the 96 wells fractions collected are printed on PATH slides with 2 printings on each slide. Positive and negative controls are printed along with the fractions. Human IgG and the unfractionated cell lysates are used as a positive control and print buffer alone is used as the negative control. Slides are stored in a sealed slide box until use.

### Microarray Assay:

The printed slides are blocked in 1% BSA in PBS+0.5% Tween 20 for 1 hour and then are rinsed with PBS+0.5% Tween 20. Human serum 1/250 dilution is incubated at 5 ug/ml in 1% BSA in PBS+0.5% Tween 20 for 2 hours. The slides are washed 3 times in PBS+0.5% Tween 20, incubated with Anti-Human IgG Alexa 647 for 1 hour, washed 3 times in PBS+0.5% Tween 20 and then rinse briefly in ddH₂O.

### Microarray Analysis:

The arrays are scanned using a Genepix scanner and the intensities of the duplicate (or triplicate) IgG binding spots detected using the Alexafluor Fluorescence dye are averaged and then baseline subtracted using the average intensity measured for the negative controls. These baseline subtracted intensities for all microarrays are then subject to Quantile normalization to eliminate any slide to slide variation followed by PAM analysis using the R Foundation for Statistical Computing ISBN 3-900051-07-0 site.

Protein identification by LC-MS/MS.

The selected protein fractions are digested with trypsin at 37° C. overnight. The resulting peptides are analyzed by LC-MS/MS using an LTQ mass spectrometer (Thermo Finnigan, San Jose, CA). Chromatographic separation of peptides is performed on a Paradigm MG4 micropump system (Michrom Biosciences Inc., Auburn, CA) equipped with a C18 separation column (0.1 mm×150 mm, C18 AQ particles, 5 μm, 120 Å, Michrom Biosciences Inc., Auburn, CA). Peptides are separated with a linear gradient of acetonitrile/water containing 0.1% formic acid at a flow rate of 300 nl/min. A 120 min linear gradient separation is used. The MS instrument is operated in positive ion mode. The ESI spray voltage is set at 2.5 KV, and the capillary voltage at 30 V. The ion activation is achieved by utilizing helium at a normalized collision energy of 35%. The data are acquired in data-dependent mode using the Xcaliber software. For each cycle of one full mass scan (range of m/z 400-2000), the three most intense ions in the spectrum are selected for tandem MS analysis, unless they appear in the dynamic or mass exclusion lists.

### Data Analysis.

All MS/MS spectra are searched against the IPI database (IPI.mouse.v3.79). The search is performed using SEQUEST algorithm version 27 incorporated in Bioworks software version 3.1 SR1 (Thermo Finnigan). The search parameters are as follows: (1) Fixed modification, Carbamidomethyl of C; (2) variable modification, oxidation of M; (3) allowing two missed cleavages; (4) peptide ion mass tolerance 1.50 Da; (5) fragment ion mass tolerance 0.0 Da; (6) peptide charges +1, +2, and +3. The identified peptides are processed by the Trans-Proteomic Pipeline (TPP)[25]. This software includes both the PeptideProphet and ProteinProphet programs. The database search results are first confirmed using the PeptideProphet software, and then the peptides are assigned for protein identification using the ProteinProphet software. In this study, both the PeptideProphet probability score and the ProteinProphet probability score are set to be higher than 0.9. This resulted in an overall false positive rate below 1% [26].

Applying the above methodology to samples derived from a human subject having suffered a severe TBI, the following proteins are observed from the slide to be present in abnormally high amounts compared to the heat mapping of non-TBI normal full brain tissue: cell adhesion molecule, tubulin beta chain, ras-related protein, synaptophysin 2, synapsin-1, receptor expression-enhancing protein, 4F2 cell-surface antigen heavy chain, TNR, neurogranin 1, secernin-1, visinin-like protein 1, neuronal cell adhesion molecule, tau, MAP6 (Microtubule associated protein), MAP1A, MAP1B, ketamine reductase, fascin 1, dematin 1 TPM1. At least six of the aforementioned elevated proteins are needed to assess the nature of the TBI as to effected tissues.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### CITED REFERENCES

1. Gerberding J L (2003). National Center for Injury Prevention and Control. Report to Congress on Mild Traumatic Brain Injury in the United States: Steps to Prevent a Serious Public Health Problem. Atlanta, GA: Centers for Disease Control and Prevention.
2. Faul, M., Xu, L. X., Wald, M. & Coronado, V. (2010) Traumatic brain injury in the United States: emergency department visits, hospitalizations, and deaths. Atlanta (GA): Centers for Disease Control and Prevention, National Center for Injury Prevention and Control.
3. Frieden, T. R., Houry, D., Baldwin, D. (2014) Report to Congress on Traumatic Brain Injury in the United States: Epidemiology and Rehabilitation. Centers for Disease Control and Prevention, National Center for Injury Prevention and Control; Division of Unintentional Injury Prevention. Atlanta, GA
4. Jager, T. E., Weiss, H. B., Coben, J. H. and Pepe, P. E. (2000) Traumatic brain injuries evaluated in U.S. emergency departments, 1992-1994. Acad Emerg Med 7, 134-140
5. Marguiles, S., Hicks, R. (2009) Combination therapies for traumatic brain injury: prospective considerations (report). J Neurotrauma 26, 925-939
6. Binder, L. M. (1997) A review of mild head trauma. Part II: Clinical implications. J Clin Exp Neuropsychol 19, 432-457
7. Ruff, R. M. and Richardson, A. M. (1999) Mild Traumatic Brain Injury. In Forensic Neurospychology: Fundamentals and Practice (Sweet, J. J., ed.), Swets & Zeitlinger, The Netherlands
8. Omalu B I, DeKosky S T, Minster R L, Kamboh M I, Hamilton R L, Wecht C H. (2005) Chronic traumatic encephalopathy in a National Football League player. Neurosurgery.; 57(1):128-34
9. Omalu B I, Hamilton R L, Kamboh M I, DeKosky S T, Bailes J. (2010) Chronic traumatic encephalopathy (CTE) in a National Football League Player: Case report and emerging medicolegal practice questions. J Forensic Nurs. 6(1):40-6
10. Omalu B. (2014) Chronic traumatic encephalopathy. Prog Neurol Surg. 28:38-49.
11. Stein T D, Alvarez V E, McKee A C (2014) Chronic traumatic encephalopathy: a spectrum of neuropathological changes following repetitive brain trauma in athletes and military personnel. Alzheimer's Research & Therapy 6:4
12. Plassman B L, Grafman J. (2015) Traumatic brain injury and late-life dementia. Handb Clin Neurol. 128:711-22.
13. Gupta R, Sen N Traumatic brain injury: a risk factor for neurodegenerative diseases. Rev Neurosci. 2015 Aug. 26. Pi August 26. pii:/j/revneuro.ahead-of-print/revneuro-2015-0017/revneuro-2015-0017.xml. doi: 10.1515/revneuro-2015-0017. [Epub ahead of print]
14. Faden A I, Loane D J. (2015) Chronic neurodegeneration after traumatic brain injury: Alzheimer disease, chronic traumatic encephalopathy, or persistent neuroinflammation? Neurotherapeutics. 12 (1):143-50.
15. Gardner R C, Burke J F, Nettiksimmons J, Goldman S, Tanner C M, Yaffe K. (2015) Traumatic brain injury in later life increases risk for Parkinson disease. Ann Neurol. 77(6): 987-95.
16. Mozzafarian D, Benjamin E J, Go A S, et al. Heart Disease and Stroke Statistics-2015 Update: a report from the American Heart Association. Circulation. 2015;131:e29-e322.
17. U.S. Cancer Statistics Working Group. United States Cancer Statistics: 1999-2012 Incidence and Mortality Web-based report. Atlanta: U.S. Department of Health and Human Services, Centers for Disease Control and Prevention and National Cancer Institute: 2015.
18. HIV Surveillance Report: Diagnoses of HIV Infection and AIDS in the United States and Dependent Areas; Division of HIV/AIDS Prevention, Centers for Disease Control and Prevention. 2013, Vol. 25
19. a. Langlois, J. A., Rutland-Brown, W. and Thomas, K. E. (2004) Traumatic brain injury in the United States: Emergency department visits, hospitalizations, and deaths. National Center for Injury Prevention and Control, Centers for Disease Control and Prevention. b. Langlois, J. A., Rutland-Brown, W. and Wald, M. M. (2006) The epidemiology and impact of traumatic brain injury: a brief overview. J Head Trauma Rehabil 21:375-378.
20. Kabadi S V, Faden A I (2014) Neuroprotective strategies for traumatic brain injury: improving clinical translations. Int. J. Mol. Sci. 15 (1): 1216-1236
21. Masel, B. E., and DeWitt, D. S. (2010). TBI: A disease process not an event. J. Neurotrauma 27 (8), 1529-40.
22. Daneshvar, D. H., Goldstein, L. E., Kierna, P. T., Stein, T. D., & McKee, A. C. (2015) Post-traumatic neurodegeneration and chronic traumatic encephalopathy. Mol. Cell Neurosci., doi:10.1016/j.mcn.2015.03.007
23. Kobeissy F, Moshourab R A. Autoantibodies in CNS Trauma and Neuropsychiatric Disorders: A New Generation of Biomarkers. In: Kobeissy F H, editor. Brain Neurotrauma: Molecular, Neuropsychological, and Rehabilitation Aspects. Boca Raton (FL): CRC Press; 2015. Chapter 29.
24. Diamond B, Honig G, Mader S, Brimberg L, Volpe B. T. (2013) Brain-reactive antibodies and disease. Annual Review of Immunology. 31:345-385.
25. Cohen I R (2013) Autoantibody repertoires, natural biomarkers, and systems controllers. Trends in Immunology. Vol. 34, No. 12, 620-625
26. Robinson W H, DiGennaro C, Hueber W, Haab B B, Kamachi M, Dean E J, Fournel S, Fong D, Genovese M C, Neuman de Vegvar, Skriner K, Hirschberg D L, Morris R I, Muller S, Pruijn G J, Van Venrooij W J, Smolen J S, Brown P O, Steinman L, Utz P J (2002) Autoantigen microarrays for multiplex characterization of autoantibody responses. Nature Medicine, Vol. 8, No. 3, 295-301.
27. Quintana, F. J. et al. (2008) Antigen microarrays identify unique serum autoantibody signatures associated with different clinical forms and pathologic subtypes of multiple sclerosis. Proc. Natl. Acad. Sci. U.S.A. 105, 18889-18894
28. Quintana, F. J. et al. (2012) Antigen microarrays identify CNS-produced autoantibodies in RRMS. Neurology 78, 532-539
29. Graham, K. L. et al. (2004) High-throughput methods for measuring autoantibodies in systemic lupus erythematosus and other autoimmune diseases. Autoimmunity 37, 269-272
30. Fattal, I. et al. (2010) An antibody profile of systemic lupus erythematosus detected by antigen microarray. Immunology 130, 337-343
31. Li, Q. Z. et al. (2005) Identification of autoantibody clusters that best predict lupus disease activity using glomerular proteome arrays. J. Clin. Invest. 115, 3428-3439
32. Hagedorn, P. H. et al. (2010) Chronic rejection of a lung transplant is marked by a profile of specific autoantibodies. Immunology 130, 427-435
33. Hagedorn, P. H. et al. (2010) Integrative analysis correlates donor transcripts to recipient autoantibodies in primary graft dysfunction after lung transplantation. Immunology 132, 394-400
34. Merbl, Y. et al. (2009) A systems immunology approach to the host-tumor interaction: large-scale patterns of natural autoantibodies distinguish healthy and tumor-bearing mice. PLOS ONE 4, e6053
35. Keller A, Nesvizhskii A I, Kolker E, Aebersold R, Empirical statistical model to estimate the accuracy of peptide identifications made by MS/MS and database search. Anal Chem 74 (2002) 5383-5392.
38. Nesvizhskii A I, Keller A, Kolker E, Aebersold R, A statistical model for identifying proteins by tandem mass spectrometry. Anal Chem 75 (2003) 4646-4658.
39. Zhiqun Zhang, J. Susie Zoltewicz, Stefania Mondello, Kimberly J. Newsom, Zhihui Yang, Boxuan Yang, Firas Kobeissy, Joy Guingab, Olena Glushakova, Steven Robicsek, Shelley Heaton, Andras Buki, Julia Hannay, Mark S. Gold, Richard Rubenstein, Xi-chun May Lu, Jitendra R. Dave, Kara Schmid, Frank Tortella, Claudia S. Robertson, Kevin K. W. Wang, Human Traumatic Brain Injury Induces Autoantibody Response against Glial Fibrillary Acidic Protein and Its Breakdown Products. PLOS One. 2014; 9 (3): e92698. Published online 2014 Mar. 25. doi: 10.1371/journal.pone.0092698

The invention claimed is:

1. A system for diagnosing traumatic brain injury (TBI) by measuring a biological sample, said diagnostic system comprising:
a proteome biochip comprising:
a glass slide with a plurality of micro-wells;
a set of brain protein fractions comprising primary serum autoantibodies reactive with brain protein autoantigens released by the TBI; and
wherein the set of brain protein fractions are printed into the plurality of micro-wells of the glass slide with a microarray printer, said biochip hybridized with control non-TBI and TBI-injured human serum samples from which an autoantibody response profile is generated;
labeled IgG or IgM secondary antibodies for addition to the plurality of micro-wells of the biochip, the labeled IgG or IgM secondary antibodies able to bind with one of the primary serum autoantibodies;
a side illumination laser to read one or more of the plurality of micro-wells in which the labeled IgG or IgM secondary antibodies bound with one of the primary serum autoantibodies; and
an optical or colormetric readout detection system for the set of brain protein fractions to screen for autoantigens present in one or more of the plurality of micro-wells that contain the labeled IgG or IgM secondary antibodies bound with one of the primary serum autoantibodies to generate a heat map to detect a relative abundance of at least six of: cell adhesion molecule, tubulin beta chain, ras-related protein, synaptophysin 2, synapsin-1, receptor expression-enhancing protein, 4F2 cell-surface antigen heavy chain, TNR, neurogranin 1, secernin-1, visinin-like protein 1, neuronal cell adhesion molecule, tau, MAP6 (Microtubule associated protein), MAP1A, MAP1B, ketamine reductase, fascin 1, dematin 1 TPM1.

2. The diagnostic system of claim 1 wherein said brain protein fractions are obtained from non-treated trauma injured brain proteomes and TBI drug treated trauma injured brain proteomes.

3. The diagnostic system of claim 1 wherein the biochip is a whole brain proteome microarray biochip.

4. The diagnostic system of claim 1 wherein the biochip displays all proteins expressed in a brain at a particular time.

5. The diagnostic system of claim 1 wherein the autoantigens are proteins present in a patient's body after a TBI.

6. The diagnostic system of claim 1 wherein the biological sample is obtained from one of blood, plasma, serum, saliva, or urine of a patient.

7. A method for diagnosing traumatic brain injury comprising:
providing the diagnostic system of claim 1;
applying a sample from a patient to the biochip;
adding an IgG or IgM secondary antibody to the biochip, the IgG or IgM secondary antibodies binding with one of the primary serum autoantibodies printed into the plurality of micro-wells of the glass slide; and
placing the biochip in the optical or colormetric readout detection system;
scanning the biochip using the side illumination laser to read one or more of the plurality of micro-wells in which the IgG or IgM secondary antibodies bound with one of the primary serum autoantibodies;
screening for the autoantigens present in one or more of the plurality of micro-wells that contain the labeled IgG or IgM secondary antibodies bound with one of the primary serum autoantibodies to generate a heat map.

8. The method of claim 7 wherein the patient has sustained a brain injury.

9. The method of claim 7 wherein diagnosing traumatic brain injury further comprises selectively detecting and measuring TBI proteome-specific autoimmune response biomarker signature panels.
